(19) United States
(12) Patent Application Publication
(10) Pub. No.: US 2013/0046393 A1
(43) Pub. Date: Feb. 21, 2013

(54) BONE IMPLANT FOR PATIENT WITH LOW BONE MINERAL DENSITY

(71) Applicants: Tzer-Min LEE, Tainan City (TW); Kuan-Chen KUNG, Tainan City (TW); Shih-Ping YANG, Tainan City (TW); Chyun-Yu YANG, Tainan City (TW); Kuo YUAN, Tainan City (TW)

(72) Inventors: Tzer-Min LEE, Tainan City (TW); Kuan-Chen KUNG, Tainan City (TW); Shih-Ping YANG, Tainan City (TW); Chyun-Yu YANG, Tainan City (TW); Kuo YUAN, Tainan City (TW)

(73) Assignee: NATIONAL CHENG KUNG UNIVERSITY, Tainan City (TW)

(21) Appl. No.: 13/477,324

(22) Filed: May 22, 2012

(30) Foreign Application Priority Data
Aug. 17, 2011 (TW) 100129396

(51) Int. Cl. A61F 2/28

(52) U.S. Cl. 623/23.55; 623/23.53

(57) ABSTRACT

A bone implant for a patient with low bone mineral density is disclosed, which includes a strontium element. The bone implant also can further include a calcium element and a phosphorus element. The strontium element is contained in a range from 0.01% mol to 99.98% mol. The calcium element is contained in a range from 0.01% mol to 99.98% mol. The phosphorus element is contained in a range from 0.01% mol to 99.98% mol.

### CROSS REFERENCE TO RELATED APPLICATION

This application claims the benefits of the Taiwan Patent Application Serial Number 100129396, filed on Aug. 17, 2011, the subject matter of which is incorporated herein by reference.

### BACKGROUND OF THE INVENTION

1. Field of the Invention

The present invention relates a bone implant and more particularly, to a bone implant for a patient with low bone mineral density.

2. Description of Related Art

Osteoporosis is the second most-significant health issue in Taiwan. Based on present records in Taiwan's Bureau of National Health Insurance, osteoporosis in 50 or more-year-old adults in Taiwan has been diagnosed in a gradually increased ratio year by year. Notably, occurrence of bone fractures in different parts of bodies increases with a decrease in bone mineral density. Recent research has reported that strontium tablets could promote osteoblast differentiation and inhibit osteoclast absorption and thus such tablets are clinically used in treatment of osteoporosis. For example, strontium ranelate developed from SERVIER co. (France), is a medicine that is taken by patients suffering osteoporosis.

In addition to osteoporosis, joint degeneration may also be aggravated with a decrease in bone mineral density, and it generally occurs at knee joints, hip joints, and vertebra. From a biological point of view, a femur in which the neck thickens is able to bear greater stress so that bone fractures do not easily occur if a patient falls down. Conversely, a femur in which the neck thins is unable to bear greater stress so that bone fractures easily occur if a patient falls down. However, an increase in thickness in a femur neck incurs greater hardness and thus makes the femur neck lose buffer function and force absorptive capacity, resulting in cartilage tissues of the femur having to be responsible for force absorption and buffer function. Therefore, it is easy to accelerate cartilage abrasion and to bring on joint degeneration. At the contact surface between two bones in a joint, a joint cartilage is located to prevent joint abrasion and damage and to introduce cushioning effects. However, a joint cartilage injured owing to aging or a trauma causes abnormal abrasion and pain. When the joint cartilage is totally abraded, direct abrasion between two bones produces bone fragments, leading to swelling and inflammation of the joint. Patients' pain in this condition is aggravated severely and joint deformation is incurred, resulting in difficult action of the joint. Accordingly, surgery for replacing hard tissues is required.

Currently, Ti alloys are a major material of hard tissues applied clinically. Because Ti alloys have mechanical strength appropriate to reduce stress transference, good anti-corrosion, and fatigue strength sufficient to be responsible for circle stress loading on a hip joint, they are considered as an excellent substitute for hard tissues. However, since strong bonding and good fixation can not occur between Ti alloys and bone tissues, a bioactive ceramic coating has to be formed on Ti alloys.

Considering that the aforesaid material requires being biocompatible to animal bone tissues, animal bone tissues are analyzed. It is found that major chemical elements contained in bone tissues are calcium ions and phosphate ions. Accordingly, hydroxyapatite (HA, Ca₁₀(PO₄)₆(OH)₂) was tested in vitro and in vivo and confirmed that formation of HA coatings on Ti alloy implants can induce osteogenesis and accelerate rapid fixation between bone tissues and the implants. In addition, HA coatings have excellent biocompatibility, osteoconductivity, and osteointegration, and thus can be applied as fillers for dental and orthopaedic or anaplastic surgery or formed on metal surfaces as substitutes for hard tissues. Also, based on clinical results of treatment, HA coatings implanted for ten years still have good fixation.

In addition to the aforementioned components, other ions such as Sr, Na, K, Mg, Fe, and Cl are found in animal bone tissues. In current research, other elements are used to substitute calcium ion, phosphate ion, and hydroxyl. Substitution of different ions makes HA properties (ex. crystallizability, lattice constant, and biocompatibility) change. Among materials containing substitute ions, some have been used in biology and materials science. In recent research, strontium ions were used to substitute for calcium ions of HA to form Sr-substituted HA (Sr-HA). According to in vivo research, when Sr-HA bone cement is implanted in defects of animal bones, good bone integration without formation of fiber layers indicates that Sr-HA bone cement is able to induce osteoblast proliferation to speed up bone healing.

Currently, clinical reports show that surgery for substituting hard tissue still has some kind of risks including patients' allergic reaction to implants, problems (breakage and modification) of an implant itself or second surgery, unsecured implants or components thereof, excessive abrasion, corrosion, bad orientation, dislocation, aging, and degeneration. Alternatively, implant functions are undesirably influenced by excess force, damage, bad installation, or improper treatment. Sometimes, implant fastening is reduced owing to changed force delivery, abrasion and damage of cement bases, or tissue responses to the implant. Bone fractures caused by exceeding unilateral force or reduction of bone strength, clots, slow healing, or ectopic ossification occur in patients' wounds. In addition, total abrasion of joint cartilage or joint deformation occurs in some implanted patients because these patients suffer osteoporosis. Hence, substitutes for hard tissues still have to be improved.

Accordingly, there is an urgent need to provide a novel implant to solve clinical problems in which patients suffering excessively low bone mineral density or osteoporosis are unable to be implanted. Therefore, it is beneficial for patients who suffer excessively low bone mineral or osteoporosis. Thus, failure risk of implant surgery can be reduced to raise success possibility of surgery.

### SUMMARY OF THE INVENTION

The object of the present invention is to provide a bone implant which can be used in patients who originally cannot be implanted due to extremely low bone mineral density or osteoporosis. The bone implant is able to efficiently induce osteoblast proliferation and differentiation so as to be naturally integrated with surrounding bones. Therefore, use of the bone implant can exhibit effects which exceed those of current clinical treatment to advance the time of healing and then to accelerate restoration of bone tissue defects. In addition, the bone implant of the present invention can be also used in patients who have normal bone mineral density.

In order to achieve the aforesaid object, the present invention provides a bone implant for a patient with low bone mineral density, comprising: a strontium element which is contained in a range from 0.01% mol to 99.98% mol.

The bone implant of the present invention can further comprise a calcium element and a phosphorus element which are respectively contained in a range from 0.01% mol to 99.98% mol.

Preferably, the strontium element is contained in a range from 0.04% mol to 95% mol. Terms of “low bone mineral density” or “excessively low bone mineral density” used herein refer to a bone mineral density measured by dual energy X-ray absorptiometry (DEXA) which is less than or equal to 2.5 standard deviations below that of a young adult reference population (diagnosed as osteoporosis) or between 1.0 and 2.5 standard deviations below that of a young adult reference population (diagnosed as osteopenia). The terms are defined according to World Health Organization (WHO) guidelines. Accordingly, the bone implant of the present invention can be used in patients with low bone mineral density or those suffering osteoporosis.

The bone implant of the present invention can further comprise an implant body. In a preferred example of the present invention, the strontium, phosphorus, and calcium elements construct a surface modification layer covering a surface of the implant body. The implant body is not specifically limited and can be made of at least one selected from a group consisting of metal, ceramic, and polymer. For example, the implant body can be a Ti or Ti alloy implant body and applied as an artificial bone substitute for joint cartilages, bone nails, and bone grafts, or as a dental implant such as orthodontic appliance, anchoring devices, dental roots, and microimplants.

Alternatively, the bone implant can be made of polymer such as gelatin, chitosan, hyaluronic acid, or composite frames thereof. Such frames have large pores in size of 100-500 μm, and thus tissues can develop in the pores. In addition, the pores can carry drugs such as bone inducing protein or collagen which is used to enhance osteogenesis.

In the bone implant of the present invention, compounds individually or simultaneously containing Sr, P, or Ca can be used to form the surface modification layer on the implant body by plasma spraying, sputtering, micro-arc oxidation, anodic oxidation, sol-gel method, simulated body fluid (SBF), or hydrothermal method.

In the present invention, the surface modification layer can be made of any material as long as the surface modification layer contains Sr, P, and Ca. For example, HA which contains both Ca and P can be used as Ca and P source of the surface modification layer and then processed with Sr compounds to form a Sr-containing calcium-phosphate coating used as the surface modification.

In the bone implant of the present invention, thickness of the surface modification layer is not particularly limited. The thickness of the surface modification layer is preferably in a range from 1 nm to 2 mm, and more preferably in a range from 1 μm to 1 mm. Pores of the surface modification layer are not limited in specific size, and preferably in size of 1 nm-1000 μm, more preferably in size of 1-300 μm.

It is difficult for patients suffering osteoporosis to be implanted clinically because they have lost too much bone mineral and had fragile bones. However, the present invention can overcome this problem. The aforesaid bone implant of the present invention can be constructed by powders or blocks made of Sr-containing calcium-phosphate compound. As long as the bone implant has Sr, this bone implant can be used in osteoporotic patients and efficiently stimulate osteoblast proliferation and differentiation so that integration between the implant and surrounding bones can exceed clinical effects of current treatment. Accordingly, time of bone healing can be advanced and restoration of bone tissue defects can be accelerated.

In one example of the present invention, the aforesaid bone implant is an implant where a common bone implant is coated with a bioactive surface modification layer. Therefore, the implant can efficiently inhibit osteoclast absorption and increase osteogenesis as well as improve the success rate of surgery (i.e., reducing failure risks of implant surgery). Clinically, the implant can be used in osteoporotic or osteopenia patients to efficiently stimulate osteoblast proliferation and differentiation so that integration between the implant and surrounding bones can exceed clinical effects of current treatment. Accordingly, time of bone healing can be advanced and restoration of bone tissue defects can be accelerated.

The Sr-containing calcium-phosphorus compounds of the present invention can construct orthopedic bone cement or filler materials. After a curing agent is added to the compounds, a formed crystal structure is similar to that of natural bones, has optimal porosity (1-20 μm) and good plastic property, as well as can be used to patch complicated fractures and defects of bones. Also, bone tissues can grow in the structure and thus mechanical bonding force between the implant and bones can be enhanced.

Other objects, advantages, and novel features of the invention will become more apparent from the following detailed description when taken in conjunction with the accompanying drawings.

### BRIEF DESCRIPTION OF THE DRAWINGS

FIG. 1 shows scanning electron microscope (SEM) images of bone implant surfaces in Test Example 1 of the present invention, wherein (a) denotes the bone implant of Comparative Example 1 and (b) denotes the bone implant of Example 1;

FIG. 2 shows energy dispersive spectrometer (EDS) spectra of bone implant surfaces in Test Example 1 of the present invention, wherein (a) denotes the bone implant of Comparative Example 1 and (b) denotes the bone implant of Example 1;

FIG. 3 shows SEM images of surfaces of bone implants immersed in simulated body fluid (SBF) in Test Example 1 of the present invention, wherein (a) denotes the bone implant of Comparative Example 1 and (b) denotes the bone implant of Example 1;

FIG. 4 shows results of removal torque test in Test Example 2 of the present invention;

FIG. 5 shows an SEM image of the frame surface of Example 2 according to Test Example 3 of the present invention; and

FIG. 6 shows energy dispersive spectrometer (EDS) spectrum of the frame of Example 2 according to Test Example 3 of the present invention.

### DETAILED DESCRIPTION OF THE PREFERRED EMBODIMENT

Because of the specific embodiments illustrating the practice of the present invention, one skilled in the art can easily understand other advantages and efficiency of the present invention through the content disclosed therein. The present invention can also be practiced or applied by other variant embodiments. Many other possible modifications and variations of any detail in the present specification based on different outlooks and applications can be made without departing from the spirit of the invention.

The drawings of the embodiments in the present invention are all simplified charts or views, and only reveal elements relative to the present invention. The elements revealed in the drawings are not necessarily aspects of the practice, and quantity and shape thereof are optionally designed. Further, the design aspect of the elements can be more complex.

### Example 1

### (1) Ti Substrate and Surface Pre-Treatment Thereof:

A Ti substrate (medical pure Ti or Ti alloy, Ti6A14V) was used as an implant body or microimplant body. The Ti substrate was ultra-sonicated with organic solvent and deionized water, sandblasted with Al₂O₃ (particle size: 355-425 μm, pressure: 4 kg/cm²), and then washed with an acidic solution to remove organic contaminations and oxide thereof.

### (2) Electroplating a Surface Modification Layer on the Implant Body:

Calcium acetate was used as a Ca-containing compound. Strontium hydroxide was used as a Sr-containing compound. Ammonium dihydrogen phosphate was used as a P-containing compound. However, in the present invention, the Ca-, Sr-, and P-containing compounds are not limited to those mentioned above. One skilled in the art can understand that other similar Ca-, Sr-, and P-containing compounds can also be used.

The aforesaid compounds were dissolved in deionized water to form an electrolyte in which the Ca- and Sr-containing compounds could be in a ratio of 2:1 and the P- and Sr-containing compounds could be in a ratio of 4.6:1. However, in the present invention, the ratio of Ca-, Sr-, and P-containing compounds is not limited to those mentioned above. One skilled in the art can understand that other ratios in the scope of the following claims can also be used.

The implant body was immersed in the electrolyte and electroplated at different voltages. During electroplating, the reaction was maintained with a circulating cooling system at a constant temperature. After electroplating, the bone implant was washed with organic solvents and deionized water, oven-dried at 60° C., and then dry-heat-sterilized at 180-200° C.

### Example 2

### (1) Synthesis of Sr—Ca—P Gelatin Frame by Co-Precipitation:

In this example, a Sr—Ca—P gelatin frame was synthesized by co-precipitation. The steps were as follows.

Titrating and titrated solutions were prepared. To the titrated solution gelatin and Ca(NO₃)₂.4H₂O were added. To the titrating solution gelatin and (NH₃)₂HPO₄ were added. During titration, Ca-containing gelatin solution was titrated with P-containing gelatin solution via a peristaltic pump at 3.8 mL/min and evenly stirred at a constant rate until the ratio of CaP/(geltin+CaP) reached 30%, 50%, and 66.6%. The formed frames were respectively named as G-30CaP, G-50CaP, and G-66.6CaP and Ca/P ratio was 1.67.

After titration, the Sr—Ca—P gelatin frames were postcured at 40° C., refrigerated at −20° C., and then lyophilizated. Therefore, in gelatin (natural polymer), nano-Sr—Ca—P compounds having unidirectional pores were directly synthesized in amounts of 30 wt %, 50 wt %, and 66.6 wt %. Accordingly, the Sr—Ca—P gelatin frames with different ratios were completed.

According to the method of this example, frames, blocks, and surface modification layers coating a common implant can be prepared.

### Comparative Example 1

### (1) Ti Substrate and Surface Pre-Treatment Thereof:

The manner of pre-treatment was the same as described in Example 1.

### (2) Electroplating a Surface Modification Layer on the Implant Body:

The manner of the example was the same as described in Example 1 except only Ca- and P-containing compounds were dissolved in deionized water to prepare an electrolyte in which the ratio of Ca- and P-containing compounds was the same as that of Example 1. The implant body was immersed in the electrolyte and electroplated at different voltages. During electroplating, the reaction was maintained with a circulating cooling system at a constant temperature. After electroplating, the bone implant was washed with organic solvents and deionized water, oven-dried at 60° C., and then dry-heat-sterilized at 180-200° C.

### Test Example 1

### Bioactivity Test

Bioactivity was used to assess whether a bone-like apatite layer formed between surfaces of bone implants and bones and then was chemically bonded and tightly bound with bones. Accordingly, bioactivity was a very important index. Based on Japan researchers' reports, if fiber-like precipitates were formed on bone implants which were immersed in simulated body fluid (SBF), the bone implant was consider as having bioactivity. Hence, the bone implants of Example 1 and Comparative Example 1, according to the present invention, were immersed in SBF for different periods of time and then taken out. The surface of the treated bone implants was observed by a scanning electronic microscope (SEM) and analyzed for element composition by an energy dispersive spectrometer (EDS) and for roughness by white light interferometers. Finally, an X-ray Diffractometer was used to determine whether apatite formed.

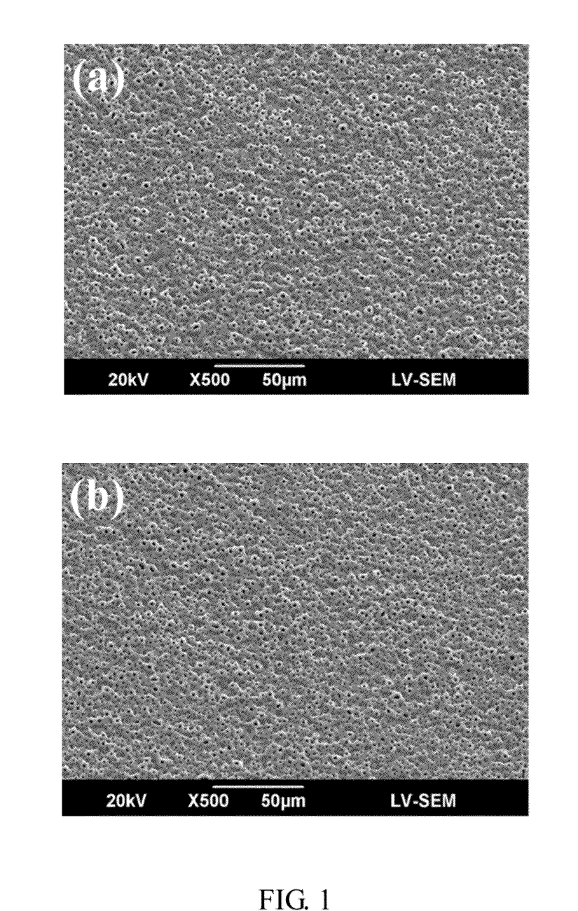

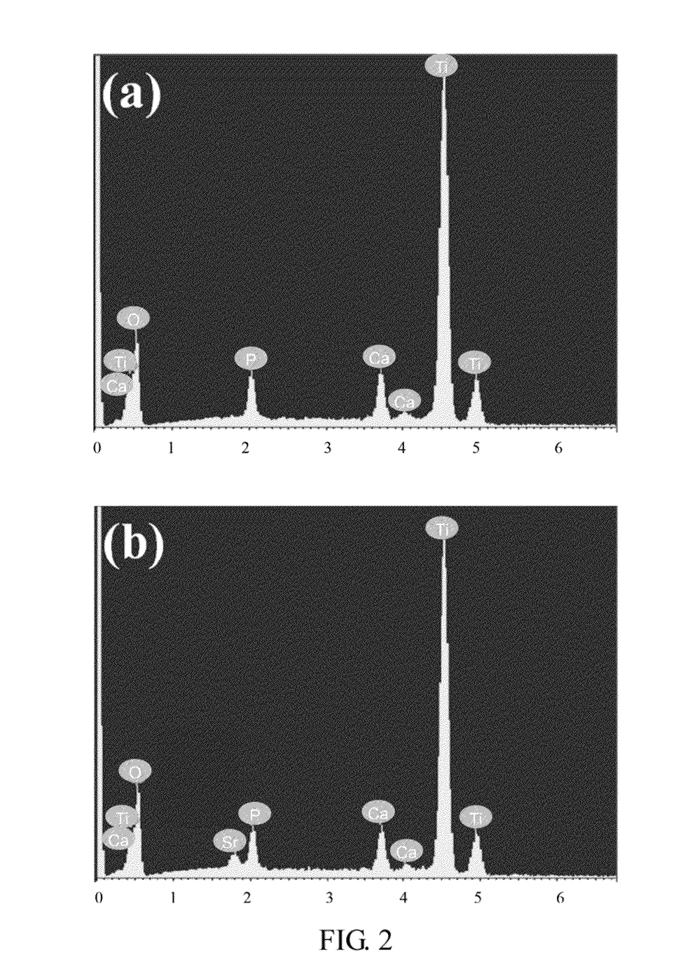

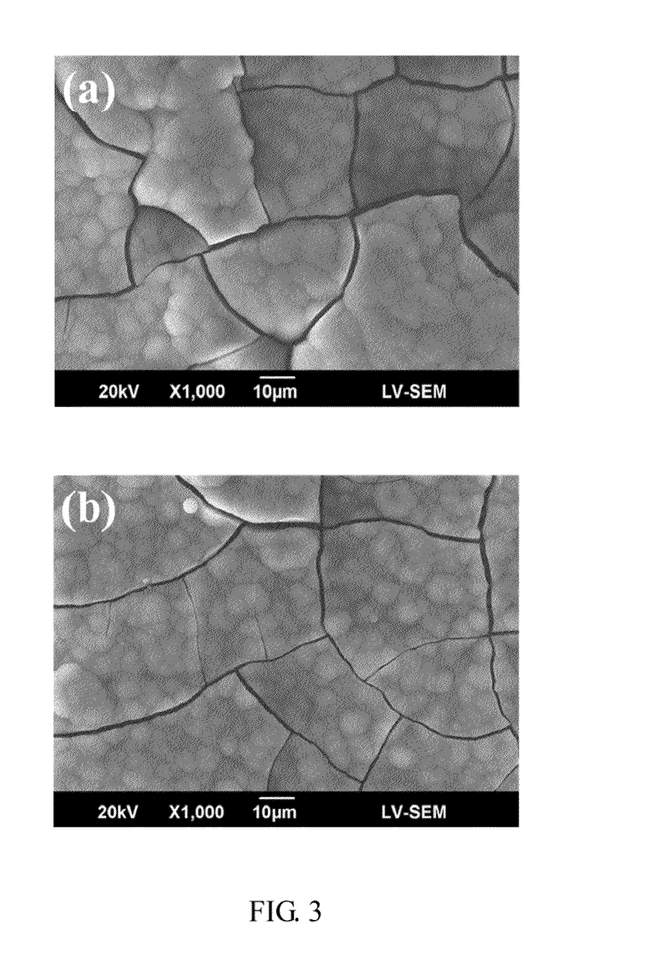

The results are shown in FIGS. 1 to 3. FIG. 1 shows SEM images of bone implant surfaces, wherein (a) denotes the bone implant of Comparative Example 1 and (b) denotes the bone implant of Example 1. FIG. 2 shows EDS spectra of bone implant surfaces, wherein (a) denotes the bone implant of Comparative Example 1 and (b) denotes the bone implant of Example 1. FIG. 3 shows SEM images of surfaces of bone implants immersed in SBF for 14 days, wherein (a) denotes the bone implant of Comparative Example 1 and (b) denotes the bone implant of Example 1.

FIGS. 1 (a) and (b) respectively represent the bone implants of Comparative Example 1 and Example 1 and they both have surface modification layers which are three-dimensional structures and have uniform distribution of pores. In addition, micro-structures on the bone implants of Example 1 and Comparative Example 1 do not have significant differences.

FIGS. 2 (a) and (b) respectively represent the element compositions of the surface modification layers on the bone implants of Comparative Example 1 and Example 1. FIG. 2(a) indicates that the surface modification layer on the bone implant of Comparative Example 1 has Ca, P, O, and Ti signals. FIG. 2(b) indicates that the surface modification layer on the bone implant of Example 1 has a Sr signal in addition to Ca, P, O, and Ti signals. These results show that the compositions of the surface modification layers on the bone implants of Example 1 and Comparative Example 1 are actually different.

When the bone implants are embedded in a human body, Ca and P can be attracted and precipitated on the surface of the bone implants and then mineralized to form chemical bonding to bones (named as bioactivity). The chemical bonding to bones is able to desirably affect osteointegration. Therefore, the index that the surface of the bone implant has bioactivity is very important, and the formation of fiber-like apatite represents bioactivity. FIGS. 3(a) and (b) respectively show the surfaces of the bone implants of Comparative Example 1 and Example 1 which both are immersed in SBF for 14 days. It can be seen that there are no microstructures (pores) on the surface of the bone implants and bone-like apatite in the form of fibers covers the surface of the bone implants. This result demonstrates that the surface modification layers on the bone implants of Example 1 and Comparative Example 1 both have bioactivity and are able to form chemical bonding to bones so as to increase stability between the implants and bones.

### Test Example 2

### Aminal Test in Osteoporotic Model

Five-month old New Zealand white rabbits (about 4-4.5 kg) were classified into two groups. The rabbits of an experimental group were treated by oophorectomy and then fed a low calcium diet to induce osteoporosis. The other group was a control group. After ten weeks, the sterilized bone implants of Example 1 and Comparative Example 1 were embedded in rabbit tibia.

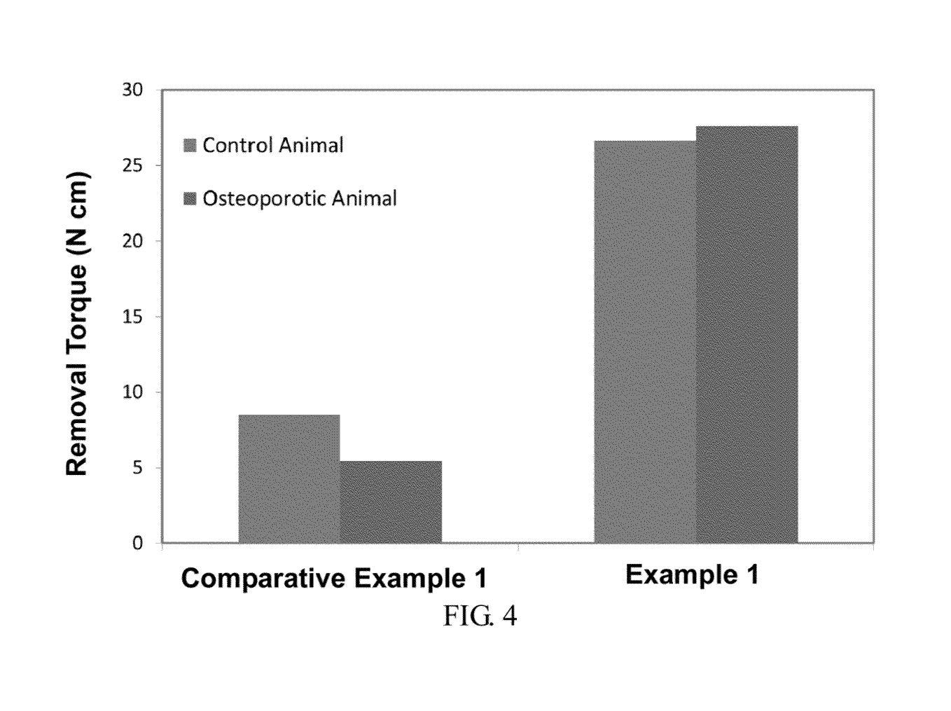

Then, histomorphometric analysis was performed to check that the tibia of the control group was a normal bone and that of the experimental group was an osteoporotic bone, and also to observe the appearance and the contact area between bones and the implants. The bone mineral density was measured by micro computed tomography (micro CT). Finally, mechanical properties, such as bonding strength and interaction, between bones and the implants were determined by removal torque analysis. The result is shown in FIG. 4.

According to the results of histomorphometric analysis, it can be confirmed that the appearance of the tibias between the control and experimental groups are significantly different, and many cavities are found in dermal bones of the tibia of the experimental group (ovary-removed rabbits). In addition, compared with the control group, the number of osteoblasts significantly decreases in the experimental group. According to the results of micro CT, it can be confirmed that bone mineral density of the experimental group also significantly decreases. These results demonstrate that osteoporosis is successfully induced in rabbits which are treated by oophorectomy and then fed a low calcium diet for two months.

Removal torque analysis was performed to check bonding strength between the bone implants and bones. The result is shown in FIG. 4. In FIG. 4, it can be seen that the bone implant of Example 1 has removal torque much higher than that of Comparative Example 1. Based on the result of Comparative Example 1 in FIG. 4, it can be understood that clinically, loosening of the bone implant occurs easily in the implanted osteoporotic patients due to low bone mineral density. Conversely, even when the bone implant of Example 1 according to the present invention is used in osteoporotic patients, its removal torque still remains at a level slightly higher than that of the control. This evidences considerably increased bonding strength between patients' bones and the bone implant of Example 1 of the present invention. Therefore, the use of the bone implant of the present invention can solve clinical problems.

### Test Example 3

### Surface Morphology Analysis and Chemical Composition Analysis

The implant prepared according to Example 2 of the present invention was observed for surface morphology by a scanning electronic microscope (SEM), and analyzed for element composition by an energy dispersive spectrometer (EDS) as well as for surface roughness by white light interferometers. Finally, an X-ray diffractometer was used to identify whether apatite forms.

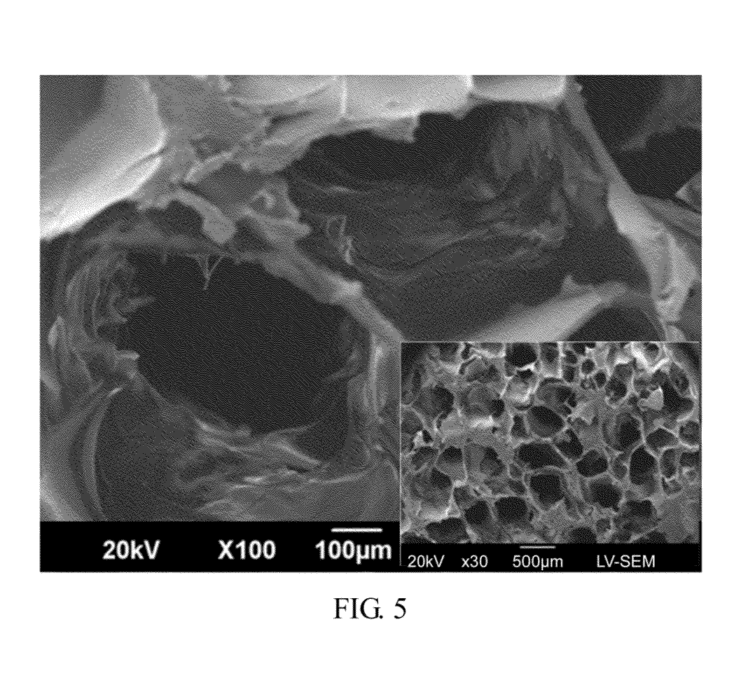

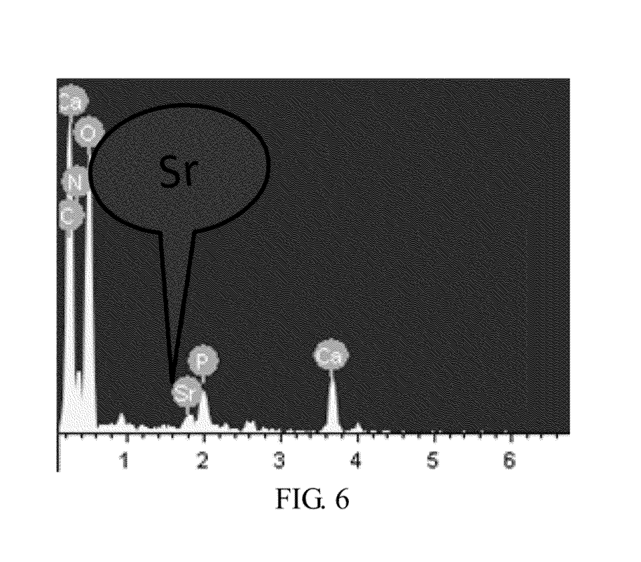

The results are shown in FIGS. 5 and 6. FIG. 5 shows an SEM image of the frame surface of Example 2. FIG. 6 shows an energy dispersive spectrometer (EDS) spectrum of the frame of Example 2. According to FIG. 5, it can be observed that pores change from a circle form to a sheet form and increase in size from 200 μm to 500 μm as the amount of the Sr—Ca—P compound increases. Based on the results of the X-ray diffractometer and EDS (FIG. 6), it can be understood that the synthesized Sr—Ca—P compound mainly consists of amorphous HA and has good osteoconductivity. The crystal observed by a transmission electron microscope (TEM) is in size about 150 nm and forms needle-posts having low crystallinity. Similar to natural bones of human bodies, the crystal has orientation and pores are suitable for osteoblast growth.

Although the present invention has been explained in relation to its preferred embodiment, it is to be understood that many other possible modifications and variations can be made without departing from the spirit and scope of the invention as hereinafter claimed.

1. A bone implant for a patient with low bone mineral density, comprising: a strontium element which is contained in a range from 0.01% mol to 99.98% mol.

2. The bone implant of claim 1, further comprising: a calcium element and a phosphorus element which are respectively contained in a range from 0.01% mol to 99.98% mol.

3. The bone implant of claim 2, further comprising: an implant body.

4. The bone implant of claim 3, wherein the strontium, phosphorus, and calcium elements construct a surface modification layer covering a surface of the implant body.

5. The bone implant of claim 4, wherein the implant body is made of at least one selected from a group consisting of metal, ceramic, and polymer.

6. The bone implant of claim 5, wherein the polymer is at least one selected from a group consisting of gelatin, chitosan, and hyaluronic acid.

7. The bone implant of claim 1, wherein the strontium element is contained in a range from 0.04% mol to 95% mol.

8. The bone implant of claim 4, wherein thickness of the surface modification layer is in a range from 1 nm to 2 mm.

9. The bone implant of claim 4, wherein the surface modification layer has pores in size of 1 nm-1000 μm.

10. The bone implant of claim 1, which is used for patients suffering osteoporosis.

11. The bone implant of claim 10, which is an artificial bone substitute or implant.
